(19) United States
(12) Patent Application Publication
(10) Pub. No.: US 2026/0248381 A1
(43) Pub. Date: Aug. 27, 2026

(54) DEVICE FOR PERFORMING OPTICAL COHERENCE TOMOGRAPHY

(71) Applicant: Heidelberg Engineering Gmbh, Heidelberg (DE)

(72) Inventors: Frank Karlheinz MÜLLER, Speyer (DE); Jörg FISCHER, Dossenheim (DE); Christoph BROSCHE, Eppelheim (DE)

(73) Assignee: Heidelberg Engineering Gmbh, Heidelberg (DE)

(21) Appl. No.: 18/870,710

(22) PCT Filed: Apr. 14, 2023
(86) PCT No.: PCT/EP2023/059756

§ 371 Date: Dec. 2, 2024

(30) Foreign Application Priority Data
Jun. 1, 2022 (DE) 10 2022 113 798.8

(51) Int. Cl. A61B 3/10; A61B 3/00

(52) U.S. Cl. CPC A61B 3/102; A61B 3/0058; A61B 3/1005

(57) ABSTRACT

In view of the problem of specifying a device for performing optical coherence tomography which is capable of reliably detecting the dimensions of an object or of structures of an object during an image capture of said object or of said structures and which has a structure that is as simple as possible, a device (10) for performing optical coherence tomography, comprising an interferometer with a light source (1), a reference arm (4) and a sample arm (5), wherein the light transmitted by the light source (1) can be split by a beam splitter (6a) such that first light is able to be guided on the reference arm (4) and second light is able to be guided on the sample arm (5), wherein the first light and the second light are able to be brought into interference and wherein a detection apparatus (3) for detecting and processing signals of the interfering first light and second light is arranged, is characterized in that an optical element (2) is arranged and is capable of further splitting the first light in the reference arm (4) into primary light (I) and secondary light (II), wherein the primary light (I) is able to be guided on a first light path (4a) and wherein the secondary light (II) is able to be guided on a second light path (4b), the length of which differs from that of the first light path (4a).

The invention relates to an apparatus according to the preamble of claim 1.

The term optical coherence tomography (usually abbreviated by OCT) is understood to be an imaging method. With this method, two-dimensional and three-dimensional images can be obtained from light-scattering structures.

In this method, light with a specific bandwidth is typically split into two partial beams in a beam splitter. The first partial beam is incident on the sample or object to be examined, the second partial beam passes through a reference section.

The light reflected by the sample or object interferes with the reference beam. Signals from the interference can be used to examine the sample with a depth resolution, i.e. in the depth of the optical axis of the first partial beam, by means of what are known as A-scans.

In addition, it is possible to also scan the surface of the sample or to scan the sample laterally with the first partial beam in order to obtain OCT images.

In OCT imaging at a posterior segment of an eye, lateral scaling of the images is often known merely as the angle of view. For converting the angle of view into absolute lengths or distances, information about the eye length and about the optical properties of the examined individual eye is necessary. In addition, knowledge of the eye length is necessary if the aim is to correct an OCT image in order to present it to scale with a correct curvature.

Against this background, primarily two ways of determining eye length are currently known. An estimation of the eye length can be made by way of a set refraction and a manually entered radius of the cornea using an eye model. It is also possible to take a direct measurement of the eye length using an appropriate device. Against this background, biometric devices are known that simultaneously record a rough image of the retina, for which the scaling can be determined with the eye length determined by them. However, these images are not suitable for diagnostic purposes due to their low quality.

A separate measurement of the eye length or corneal curvature constitutes additional effort for a user. Therefore, these measurements are not always performed, or the results are not transferred to software for evaluating OCT images of the posterior segment of the eye. If the values are entered and the entry is done manually, transmission errors may also occur. The additional integration of the technology of a biometer into a diagnostic retina OCT device would significantly increase system complexity.

The invention is therefore based on the object of providing an apparatus for carrying out optical coherence tomography with which, during acquisition of an image of an object or of structures of an object, its or their dimensions can be reliably measured with the simplest possible construction.

The present invention achieves the aforementioned object by way of the features of claim 1.

Such an apparatus comprises an interferometer having a reference arm and a sample arm, wherein the respective light guided along these arms may be made to interfere with the respective other light, and wherein a detection device detects and processes signals of the interference.

According to the invention, an optical element is provided with which the light in the reference arm may be split or divided further, wherein primary light may be guided along a first light path of the reference arm and wherein secondary light may be guided along a second light path of the reference arm, the length of which differs from that of the first light path.

According to the invention, such an OCT apparatus with an interferometer having a light-splitting optical element can be used to simultaneously or substantially simultaneously record OCT images at different distances from the apparatus. According to the invention, the distance of a camera or an objective of the apparatus from the object, in particular from the eye, can also be measured simultaneously or substantially simultaneously with the imaging of a portion of an object, preferably of the posterior segment of the eye. This allows the length of an object or the lengths of structures of the object, in particular the optical eye length, to be determined very precisely.

A detection device could, in particular as part of a spectrometer, detect and process the respective signals generated by the primary or secondary light of the respective first or second light path separately from one another and generate an A-scan simultaneously or with a short time interval for both the first and second light paths. As a result, an A-scan of the retina and cornea can be created simultaneously with each camera frame. No error-prone algorithms are required to register overlapping structures in an image.

Against this background, light of a first spectral range could be guidable along the first light path and light of a second spectral range could be guidable along the second light path. As a result, light is split into light beams from different spectral ranges, i.e. into light beams having different wavelengths. Thus, signals of at least two spectral ranges can be recorded by a line-scan camera of the detection device in the detector arm of the interferometer and processed separately for the two spectral ranges. This allows OCT images to be recorded simultaneously or substantially simultaneously in two spectral ranges at different distances from the apparatus.

The spectral ranges of the light paths might not overlap each other or might overlap only slightly. Alternatively, the spectral ranges of the light paths could be separated from one another by a wavelength range or spectral range. The line-scan camera can detect light wavelengths from a first interval largely or completely separately from light wavelengths from another interval particularly well with a good signal quality.

Light of a second spectral range could be guidable along the second light path, wherein light of in each case two spectral ranges may be guided along the first light path, the latter two spectral ranges being separated from each other by a wavelength range. The line-scan camera can thus detect light wavelengths from two different, separate first intervals, which are separated from light wavelengths from a second interval, particularly well. The two spectral ranges of the first light path might not overlap or overlap only slightly with the spectral range of the second light path. A very good, or sufficiently good, signal quality can thus be achieved.

Using the detection device, the distances of structures of an object from the apparatus could be ascertainable simultaneously, or with a short temporal distance, from the axial positions of the structures in an A-scan and from the lengths of the first and second light paths. Thus, a light path can be specifically assigned to a specific structure to be detected.

The length of the examined object and/or the distance of the structures within the object, in particular from one another or from the apparatus, could be ascertainable from the ascertained distances. Thus, an object can be examined not only with regard to its spatial extent, but even with regard to its internal spatial construction.

The length of an eye can be determined using the apparatus described herein. Alternatively or additionally, the distance between the cornea of an eye and its retina can be determined using the apparatus. These data can be used for further diagnosis by a physician.

Against this background, an image generating device could generate and/or present laterally scaled images of the retina based on the ascertained length of the eye. As a result, a physician can make their diagnosis particularly well, because all the essential information is presented to them visually in images.

The detection device could comprise a line-scan camera. Alternatively or in addition, the light source could be a broadband light source. Using a broadband light source, light of a very broad spectral range can be emitted, which may be split into light beams of different spectral ranges, with these latter spectral ranges being partial ranges of the broad spectral range. The line-scan camera allows the very selective and defined detection of light from the different spectral ranges.

Using the apparatus, it may be possible to switch between two modes of image generation, to be precise between a first mode in which images of the retina and the cornea of an eye may be recorded and/or presented simultaneously, and a second mode in which only images of the retina of the eye may be recorded and/or presented. The switchable recording mode could be realized by way of a switching device that allows switching between a spectrally split simultaneous mode and a mode that allows the full spectral bandwidth for the retinal image. In contrast to the prior art, this switching device does not switch back and forth between different z ranges.

Against this background, the optical element could be connectable into the beam path of the reference arm or be pivotable into the beam path and could be removable therefrom or be pivotable out of it. A pivoting-in process is mechanically easily implementable, for example by an electric motor, which moves the optical element about a pivot axis.

The optical element could have a beam splitter, in particular a dichroic beam splitter, or be formed as such. This allows light or a light beam to be split into two light beams that travel along different light paths. A dichroic beam splitter splits an incident light beam into light beams of different spectral ranges.

The optical element could be designed as a dichroic element or include a dichroic element. This makes it possible to split light into partial beams with different wavelengths. As a dichroic element, a dichroic mirror, which is arranged directly in the beam path of the reference arm and is reflective only for the first spectral range or the first spectral ranges of the primary light, could be used, for example, instead of a discrete beam splitter and a mirror.

The dichroic element could alternatively be designed as a dichroic lens or as a dichroic coated lens. A lens can be used together with a switching device, with which the pivoting-in or pivoting-out of a single, compact optical component must be realizable.

Furthermore, the use of a dichroic lens is advantageous in particular when an adjustment with respect to a single-mode fiber is necessary. A dichroic or dichroically coated lens could be arranged in the beam path of the reference arm, which has or exerts in total no or only a slight refraction on the transmitted light. If the light is input into the reference arm with a fiber, this has the advantage that the adjustment is facilitated, but more light is lost. An uncritical adjustment has the advantage that the lens can be pivoted into and out of the beam path easily, and thus a simple and robust switching between the modes “complete spectrum for retinal visualization” and “simultaneous presentation of cornea and retina for the determination of the optical eye length” can be realized.

The dichroic element could be designed as a dichroic mirror, which is reflective only for a defined wavelength range or spectral range. Such a mirror can reflect a defined spectral range of incident light and allow another one to pass through. An arrangement for adjusting the working distance of an objective from an object to be examined and/or for lateral adjustment of an objective could comprise an apparatus of the type described herein. The apparatus described here could be integrated in a fully automated retinal diagnostics system as a Z-sensor for an automatic adjustment device to set the correct working distance between the objective and the apex or eye apex.

The apparatus described here can be used not only for spectral domain OCTs but also for swept source OCT systems. When implementing the apparatus in a particular system, a non-contiguous range of the light spectrum could be used. This can greatly increase the spatial resolution at the expense of higher sidebands.

The beam paths and light paths described here can be implemented not only within the framework of a free-beam optical unit, but also at least partially in a fiber-optics system. Therefore, the apparatus may have optical fibers.

A measurement of the distance between the apparatus described here and the eye to be examined can be performed during the OCT image acquisition at the posterior segment of the eye in order to measure dimensions of the eye, in particular the eye length. Using the apparatus described here, no separate measurement of the eye length is necessary, and there is no source of error when transmitting data or because no data is entered. This increases the average accuracy of an absolute scaling specification of retina OCT recordings as well as of concurrently recorded cSLO images of a confocal scanning laser ophthalmoscope.

In the prior the art, corneal curvature and refraction are currently used to estimate an axial length and to thus determine a scaling. However, the determination of the axial length becomes incorrect if the eye parameters which are not taken into account, such as corneal curvature of the second surface, anterior chamber depth, and lens parameters, deviate from a model eye used. This is the case in particular when a refractive error of a patient has been corrected by using intraocular lenses (IOLs). The direct measurement of an axial length is therefore a much more robust parameter for scaling determination.

By better matching the scaling between test data and reference data, classification methods can achieve better test strength. Methods in which no dense volumes are recorded but which use scan patterns with fixed scaling also benefit from a better match between the recording location of the recorded OCT sectional images and the target positions. For example, in circular scans with fixed radius, the actual radius in the eye will vary less.

It is further advantageous that the distance measurement can be used to check during the recording whether the relative position of the camera of the apparatus to the eye is correct.

This information can be used to manually or automatically adjust the camera. The OCT signal of the retina can be used as a direct manipulated variable (retinal signal in the sweet spot for the reference arm length set optimally for eye length) for the automatic distance setting, provided the optical eye length is known.

If the eye length is not known, the automatic distance setting can be made based on the corneal signal, and the reference arm can then be set optimally for the retinal signal in a second automated step.

The position of the usable corneal signals in a B-scan can also be used to obtain information about the lateral adjustment of the camera in the scan direction.

If the scan direction is varied and differently oriented radial scans alternate, for example two orthogonal scans, then the position information is also available in 2D.

A further potential application of the apparatus is the measurement of retinal curvature, which may be relevant for various pathologies, especially in myopia patients. The curvature of the retinal signal depends on the working distance between the objective apex and the corneal apex. If the optical globe length is known, the working distance can be reliably determined from the known parameters “reference arm length” and “sample arm length up to the objective apex.” Using appropriate eye models, the true curvature of the retina can thus be determined much more accurately.

Compared to methods that sequentially measure the distance from the retina and cornea, for example by varying the reference arm length, the advantage of using the apparatus described here is that the measurement is performed simultaneously. Therefore, errors in the length measurement due to axial movements of the eye are largely excluded.

The use of the apparatus described here could offer advantages in the following points. The accuracy of classification methods that depend on scaling is increased. Manual adjustment is assisted by specifying the distance between the eyes or derived indicators, which on average results in a higher image quality. An automatic adjustment function is supported. A true-to-scale representation of the retina with the actual curvature is made possible.

### In the drawing,

FIG. 1 shows a schematic illustration of a recording of the posterior segment of the eye, in which the visual field angle is shown as device parameter and in which the size of the region recorded at the fundus d is not directly accessible because the optics and size of the examined eye are not known,

FIG. 2 shows a schematic illustration of an apparatus for performing optical coherence tomography, comprising an interferometer in which an optical element is arranged in the reference arm, wherein a first spectral range is used in a first channel of the detection device and in a second channel a second spectral range is used for image generation,

FIG. 3 shows a further schematic illustration of the apparatus for performing optical coherence tomography, in which an optical element is arranged in the reference arm, wherein in the first channel of the detection device, two non-contiguous spectral ranges at both ends of a spectrum are used for image generation, and wherein in a second channel, a second spectral range is used for image generation, and

FIG. 4 shows in the left-hand illustration a schematic illustration of a dichroic mirror arranged as an optical element in the beam path of a reference arm, and shows in the right-hand illustration a schematic illustration of a dichroically coated lens arranged as an optical element in the beam path of the reference arm.

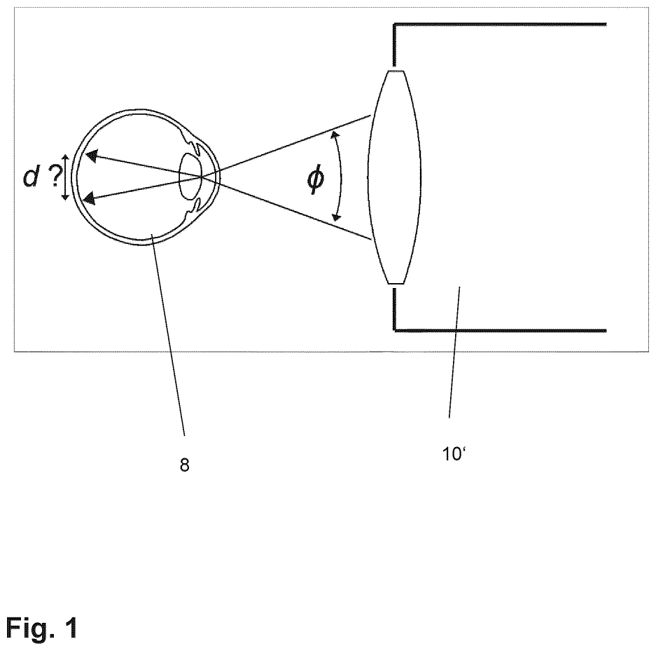

FIG. 1 shows a schematic illustration of a recording of the posterior segment of a human eye 8 by OCT or cSLO, wherein the visual field angle (is shown as a device parameter and in which the size of the recorded region at the fundus d is not directly accessible because the optics and size of the examined eye 8 are not known. An apparatus 10′ of the prior art is used for recording the posterior segment of the eye.

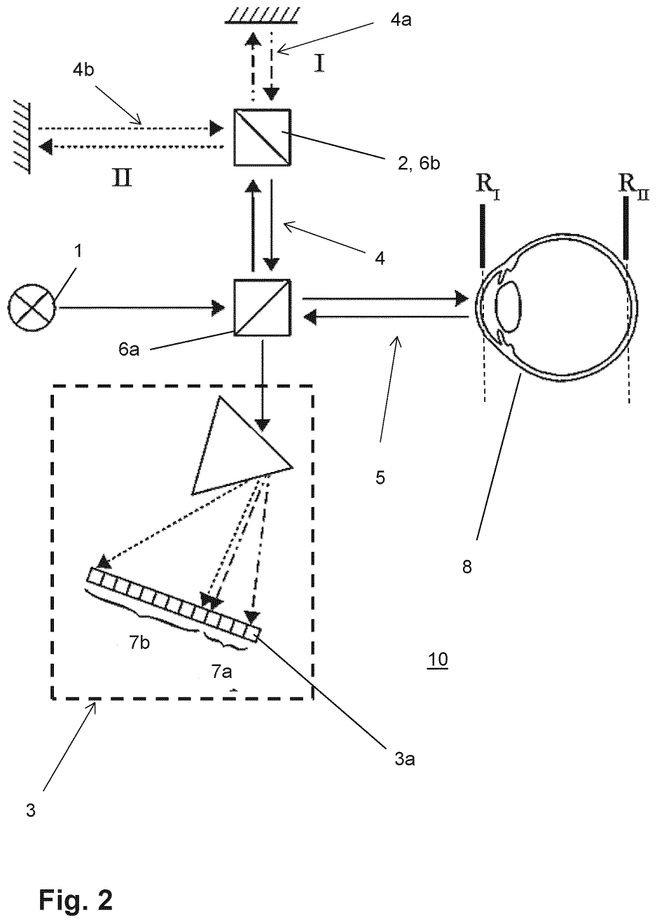

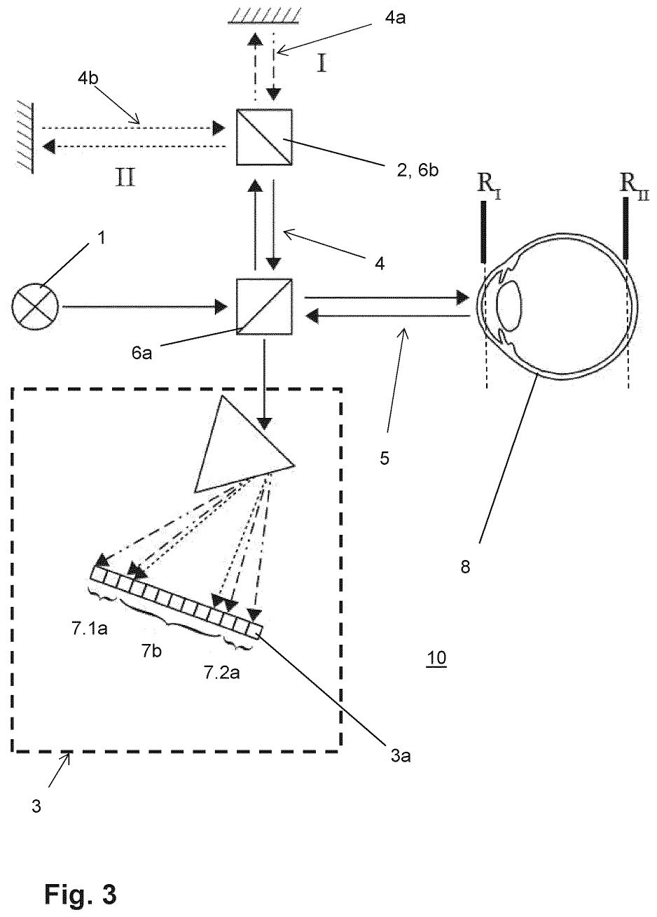

FIGS. 2 and 3 show, against this background, in each case in a schematic and partial illustration, an apparatus 10 for performing optical coherence tomography, comprising an interferometer having a broadband light source 1, a reference arm 4, and a sample arm 5, wherein the light emitted by the light source 1 is split using a beam splitter 6a such that first light is guided along the reference arm 4 and second light is guided along the sample arm 5.

The first and second light are made to interfere, and a detection device 3 is arranged for detecting and processing signals of the interfering first and second light.

An optical element 2 with which the first light in the reference arm 4 is further split into primary light (I) and secondary light (II) is arranged. The primary light (I) is guided along a first light path 4a in the reference arm 4, and the secondary light (II) is guided along a second light path 4b in the reference arm 4, the length of the second light path 4b being different from the length of the first light path 4a.

In this respect, the first light comprises primary light (I) and secondary light (II) in different light paths 4a, 4b, which interferes with the second light in each case.

In FIG. 2, the detection device 3 detects and processes the respective signals generated by the primary and secondary light (I, II) of the respective first and second light path 4a, 4b separately from one another and simultaneously or with a short time interval generates a respective A-scan for both the first light path 4a and the second light path 4b.

Primary light (I) to be detected of a first spectral range 7a is guided along the first light path 4a, and secondary light (II) to be detected of a second spectral range 7b is guided along the second light path 4b.

The spectral ranges 7a, 7b of the primary light (I) and of the secondary light (II) along the light paths 4a, 4b do not overlap one another.

The detection device 3 comprises a line-scan camera 3a. The signals for both spectral ranges 7a, 7b are recorded by the line-scan camera 3a in the detector arm of the interferometer and processed separately for the two spectral ranges 7a, 7b or even wavelength ranges. Each spectral range 7a, 7b corresponds to a wavelength range comprising light with light wavelengths from a specific interval.

This results in an A-scan of the retina and the cornea of the eye 8 simultaneously with each camera frame. No error-prone algorithms are required to register overlapping structures, for example of cornea and retina, in an image.

The absolute distance of the cornea from the apparatus 10 or from the objective of a camera of the apparatus 10 can be determined from the OCT images of the first spectral range 7a.

The optical distance of the retina from the apparatus 10 or from the objective of a camera of the apparatus 10 can be determined from the position of the retina in the OCT images for the second spectral range 7b.

The difference between the two positions of the cornea and retina is the optical length of the eye. The resulting eye length can be used to estimate or ascertain the lateral scaling of the retinal images with greater accuracy.

After all, in the reference arm 4, the broadband light from the light source 1 is split into light paths 4a, 4b of different lengths using the optical element 2 such that the length of the reference arm 4a for a first part (I) of the light spectrum corresponds to the distance of the apparatus 10 from the cornea of an eye 8, while the length of the reference arm 4b for the other, second part (II) of the light spectrum corresponds to the optical distance of the apparatus 10 from the retina.

Even with a very small wavelength range, sufficient resolution can be achieved to be able to determine the position of the cornea sufficiently well for the depth resolution of diagnostic images to be only minimally reduced.

FIG. 3 shows, against this background, the apparatus 10, in which secondary light (II) of a second spectral range 7b to be detected is guided along the second light path 4b, wherein primary light (I) of in each case two first spectral ranges to be detected 7.1a, 7.2a may be guided along the first light path 4a, wherein these latter first two spectral ranges 7.1a, 7.2a are separated from each other by a wavelength range and do not overlap with the second spectral range 7b of the second light path 4b.

Therefore, in a first channel of the detection device 3, the two non-contiguous first spectral ranges 7.1a, 7.2a at both ends of a spectrum are used for image generation.

In the second channel, the contiguous second spectral range 7b is used for image generation.

Therefore, the first channel produces a higher resolution, but stronger sidebands are created. The second channel produces a slight reduction of the signal-to-noise ratio and resolution.

Using the detection device 3 of the apparatuses 10 in FIGS. 2 and 3, the distances of structures of an object from the detection device 3 or the apparatus 10 are ascertainable simultaneously, or with a short temporal distance, from the axial positions of the structures in an A-scan and from the lengths of the first and second light paths 4a, 4b.

The length of the examined object and/or the distance of the structures within the object, in particular from one another, are ascertainable from the ascertained distances of the structures from the apparatus 10.

Using the apparatuses 10 of FIGS. 2 and 3, the length of an eye 8 and/or the distance of the cornea of an eye 8 from its retina can be determined.

Based on the ascertained length of the eye 8, an image-generating device can generate and/or present laterally scaled images of the retina.

In the apparatuses according to FIGS. 2 and 3, it is possible to switch between two modes of image generation, to be precise between a first mode in which images of the retina and the cornea of the eye 8 may be recorded and/or presented simultaneously, and a second mode in which only images of the retina of the eye 8 may be recorded and/or presented.

For this purpose, the optical element 2 is connectable into the beam path of the reference arm 4 or pivotable into the beam path and is removable therefrom or pivotable out of it.

FIGS. 2 and 3 show that the optical element 2 has a dichroic beam splitter 6b and guides the primary light (I) and the secondary light (II) in different directions along their light paths 4a, 4b.

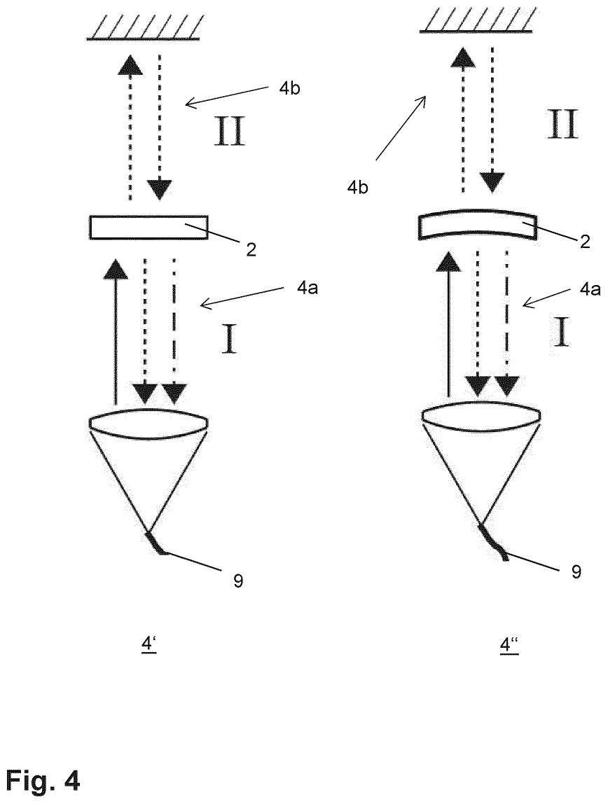

FIG. 4 shows, using two sections of alternative further reference arms 4′, 4″, that the optical element 2 is designed as a dichroic element or comprises a dichroic element.

FIG. 4 shows on the left that the dichroic element is designed as a dichroic mirror which is reflective only for a defined spectral range or wavelength range. Specifically, primary light (I) is reflected and secondary light (II) is allowed to pass through. This results in two light paths 4a, 4b of different lengths.

FIG. 4 shows on the right that the dichroic element is designed as a dichroic lens or as a dichroically coated lens. The dichroically coated lens is arranged in the beam path of the reference arm 4″, which in total exerts no or only a slight refraction on the transmitting secondary light (II).

Since the light is input into the reference arm 4″ using an optical fiber 9, adjustment is made easier. Schematically illustrated is an optical fiber 9, which can be designed in particular as a single-mode fiber.

In the OCT reference arm 4, 4′, 4″, the broadband light originating from the light source 1 is preferably split into light paths 4a, 4b of different lengths using a dichroic optical element 2 such that the length of the reference arm 4a for a first part (I) of the light spectrum corresponds to the distance of the apparatus 10 from the cornea of an eye 8, while the length of the reference arm 4b for the other, second part (II) of the light spectrum corresponds to the optical distance of the apparatus 10 from the retina.

FIGS. 2 and 3 show the optical distance plane R_{I}, in which the cornea is located, and the optical distance plane R_{II}, in which the retina is located. The distance between the distance planes R_{I}, R_{II} corresponds to the length of the eye 8.

Using the apparatuses 10 described herein, OCT imaging of the posterior segment of the eye is possible with simultaneous or substantially simultaneous measurement of the distance of the apparatus 10 from the eye 8. This is realized by using a reference arm 4 with a dichroic element.

### LIST OF REFERENCE SIGNS

1 Light source of 10
2 Optical element in 4
3 Detection device
3b Line-scan camera
4, 4′, 4″ Reference arm
4a First light path in 4, 4′, 4″
4b Second light path in 4, 4′, 4″
5 Sample arm
6a First beam splitter
6b Second beam splitter, dichroic
7a First spectral range
7b Second spectral range
7.1a First first spectral range
7.1b Second first spectral range
8 Eye
9 Optical fiber
10, 10′ Apparatus
I Primary light along 4a
II Secondary light along 4b
R_{I} Distance plane of the cornea at a distance from 10
R_{II} Distance plane of the retina at a distance from 10

1. An apparatus for performing optical coherence tomography, comprising an interferometer having a light source, a reference arm, and a sample arm, wherein the light emitted by the light source may be split using a beam splitter such that first light may be guided along the reference arm and second light may be guided along the sample arm, wherein the first and the second light may be made to interfere and wherein a detection device is arranged for detecting and processing signals of the interfering first and second light, characterized in that an optical element with which the first light in the reference arm may be split further into primary light (I) and secondary light (II) is arranged, wherein the primary light (I) may be guided along a first light path and wherein the secondary light (II) may be guided along a second light path, the length of which differs from that of the first light path

2. The apparatus as claimed in claim 1, wherein the detection device detects and processes the respective signals generated by the primary or secondary light (I, II) of the respective first or second light path separately from one another and simultaneously or with a short time interval generates an A-scan for both the first light path and the second light path.

3. The apparatus as claimed in claim 1, wherein light (I) of a first spectral range may be guided along the first light path and light (II) of a second spectral range may be guided along the second light path.

4. The apparatus as claimed in claim 3, wherein that the spectral ranges of the light paths do not overlap or overlap only slightly and/or are separated from one another by a wavelength range.

5. The apparatus as claimed in claim 1, wherein light (II) of a second spectral range may be guided along the second light path, wherein light (I) of in each case two spectral ranges may be guided along the first light path, wherein these latter spectral ranges are separated from each other by a wavelength range and/or do not overlap or overlap only slightly with the spectral range of the second light path.

6. The apparatus as claimed in claim 1, wherein the distances of structures of an object from the detection device or from the apparatus can be determined by means of the detection device simultaneously or with a short time interval from the axial positions of the structures in an A-scan and from the lengths of the first and second light paths.

7. The apparatus as claimed in claim 6, wherein the length of the examined object and/or the distance of the structures within the object, in particular from one another, is or are ascertainable from the ascertained distances.

8. The apparatus as claimed in claim 1, wherein it can be used to determine the length of an eye and/or the distance of the cornea of an eye from its retina.

9. The apparatus as claimed in claim 8, wherein an image-generating device generates and/or represents laterally scaled images of the retina on the basis of the ascertained length of the eye.

10. The apparatus as claimed in claim 1, wherein the detection device comprises a line-scan camera and/or in that the light source is broadband.

11. The apparatus as claimed in claim 1, wherein it is possible to switch between two modes of image generation, to be precise between a first mode in which images of the retina and the cornea of an eye may be recorded and/or presented simultaneously, and a second mode in which only images of the retina of the eye may be recorded and/or presented.

12. The apparatus as claimed in claim 11, wherein the optical element is connectable into the beam path of the reference arm or pivotable into the beam path and is removable therefrom or pivotable out of it.

13. The apparatus as claimed in claim 12, wherein the optical element has a beam splitter.

14. The apparatus as claimed in claim 12, wherein the optical element is designed as a dichroic element or comprises a dichroic element.

15. The apparatus as claimed in claim 14, wherein the dichroic element is designed as a dichroic lens or as a dichroically coated lens.

16. The apparatus as claimed in claim 14, wherein the dichroic element is designed as a dichroic mirror which is reflective only for a defined wavelength range.

17. An arrangement for adjusting the working distance of an objective from an object to be examined and/or for laterally adjusting an objective, comprising an apparatus as claimed in claim 1.
